(19) United States
(12) Patent Application Publication
(10) Pub. No.: US 2010/0221223 A1
(43) Pub. Date: Sep. 2, 2010

(54) IMMUNE RESPONSE INDUCTION METHOD

(75) Inventors: Yuuki Tsutsui, Tokyo (JP); Kiyo Nakanishi, Tokyo (JP); Shunsuke Watanabe, Tokyo (JP); Shigeo Takemura, Tokyo (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Astellas Pharma Inc., Tokyo (JP)

(21) Appl. No.: 12/777,202

(22) Filed: May 10, 2010

Related U.S. Application Data
(62) Division of application No. 10/674,581, filed on Sep. 29, 2003, now abandoned.
(60) Provisional application No. 60/415,026, filed on Sep. 30, 2002.

(51) Int. Cl. A61K 38/21

(52) U.S. Cl. 424/85.7

(57) ABSTRACT

The present invention proposes a method of inducing both vaccine antigen-specific antibody in blood and vaccine antigen-specific antibody secreted at the mucosal surface using vaccine antigen and adjuvant of said vaccine antigen.

### CROSS-REFERENCE TO RELATED APPLICATIONS

This application is a division of U.S. application Ser. No. 10/674,581, which application claims the benefit of priority to U.S. application Ser. No. 60/415,026, filed Oct. 4, 2002, the disclosures of which are hereby incorporated by reference in their entirety for all purposes.

### TECHNICAL FIELD

The present invention relates to a method of effectively inducing both antibodies in blood specific to various vaccine antigens and antibodies secreted at the mucosal surface specific to various vaccine antigens, a vaccine composition, a mucosal adjuvant, a combination product of vaccine antigen and mucosal adjuvant, as well as a mucosal adjuvant for inducing both vaccine antigen-specific antibody in blood and vaccine antigen-specific antibody secreted at the mucosal surface whose active ingredient is an interferon α.

In further detail, the present invention relates to a method of inducing both vaccine antigen-specific antibody in blood and vaccine antigen-specific antibody secreted at the mucosal surface using vaccine antigen and adjuvant of this vaccine antigen, comprising

(1) mucosal administration of vaccine antigen,

(2) the use of an interferon α as the active ingredient of the adjuvant,

(3) administration of this adjuvant at the same time as or at a different time than this vaccine antigen, and

(4) mucosal administration of this adjuvant by the same administration route as this vaccine antigen,

a vaccine composition, a mucosal adjuvant, a combined product of vaccine antigen and mucosal adjuvant, as well as a mucosal immune response activation method that uses mucosal adjuvant, and the like.

### PRIOR ART

Vaccines are generally classified into two types, live vaccines that use a live infectious pathogen and noninfectious inactivated vaccines in which the pathogen or toxin has been inactivated. Vaccines have thus far been used as injections for the most part. Vaccines in injection form that reach the circulatory system are known to induce systemic immune response.

It should be noted that with live vaccines it is possible to acquire immune response capability that is the most similar to that of natural immunization, but it is difficult to control pathogenicity of vaccine strains and there is concern over their safety in humans, such as a return to virulent as a result of in vivo mutation. Although inactivated vaccines are very safe when compared to live vaccines, in general, immunogenicity is weak, and there are not sufficient effects in terms of inducing secreted antibody, making practical use difficult. Therefore, attempts have been made to develop a substance that promotes induction of immunity to vaccine antigen, that is, an adjuvant, for instance, various types of immune adjuvants, including an adjuvant whose active ingredient is an inorganic salt that is used sparingly in humans, such as aluminum hydroxide, aluminum phosphate, or calcium phosphate, and cytokines, such as IL-2, 4, 12 or interferon γ (refer to patent reference 1, non-patent reference 1, non-patent reference 2, non-patent reference 3). Nevertheless, these induce a systemic immune response when made into an injection and induction is not realized at the mucous membrane surface.

Many infectious pathogens invade the body via mucous membranes covering the surface of organs that come into direct contact with the outside, such as the nasal mucous membranes, buccal mucous membranes, pulmonary mucous membranes, gastrointestinal mucous membranes, and vaginal mucous membranes. Consequently, it appears that activating immunity by antigen-specific immune response at the mucous membranes can more effectively prevent invasion of infectious pathogens. From this point, there is an abundance of research being conducted on “mucosal vaccines” as the next-generation vaccines that will replace conventional vaccines in injection form and with which mucosal administration to the above-mentioned mucous membranes is performed by an administration method such as oral, nasal, pulmonary, vaginal, and the like. It is known from recent approaches that “mucosal vaccines” induce systemic immune response, and at the same time, induce mucosal immune response; it is possible to evaluate induction of mucosal immune response by determining the IgA antibody at the mucous membrane, such as the IgA antibody in feces; the details of this mechanism of action are not known, but IgA-producing precursor cells specific to the pathogen that has invaded the mucosal tissue are not only seeded at the place of invasion, but also other mucosal sites throughout the body with blood flow, and the like; and large amounts of IgA antibody are secreted at of course the mucosal sites at the place of invasion, as well as mucosal sites outside the place of invasion and, IgA-producing precursor cells also enter the blood flow to produce IgG.

Immunogenicity of the vaccine itself is low with mucosal administration of vaccines and attempts have been made to use an adjuvant that is administered together with a vaccine given by mucosal administration, that is “a mucosal adjuvant.”

The possibility of using cholera toxin, Escherichia coli heat-labile toxin, lipopolysaccharides derived from gram-negative bacteria has been studied, but even though these are given by mucosal administration, there are concerns over safety and practical use has not been realized.

Furthermore, cytokines, and the like, have also been studied from the point of safety, and it is reported that IL-12 and interferon β have mucosal adjuvant activity (refer to patent reference 2 and patent reference 3).

A method for augmenting mucosal immunity by intranasal administration of IL-12 is disclosed in patent reference 2. The effective dose of IL-12 is cited as 0.5 μg/kg to 150 μg/kg in this reference. On the other hand, when 1 μg of IL-12 was given by nasal administration to mice for six consecutive days, 50% of the mice died (non-patent reference 4). Moreover, there are reports that bioavailability was 10 to 20% with the above-mentioned preferred dose and when IL-12 was given to mice by nasal administration (subcutaneous injection and intraperitonal administration; non-patent references 5 and 6), and adverse effects with a dose of 0.03 μg/kg to 0.5 μg/kg have been reported in human clinical trials of IL-12 subcutaneous administration (non-patent reference 7). Taking the above-mentioned into consideration, there are still safety problems with the use of IL-12 as a mucosal adjuvant. Moreover, although human clinical trials have been conducted on IL-12 intended as an anticancer and other types of drugs, it still is not a cytokine that can be used for practical purposes in humans because of the toxicity thereof.

A vaccine antigen given at the same time as or at a different time than interferon β adjuvant by administration via the nasal, buccal, or pharyngeal mucous membranes is cited in patent reference 3. Specifically, it is cited that in terms of the serum anti-tetanus toxoid antibody titer (IgG) as a result of simultaneous nasal administration of tetanus toxoid antigen and interferon β adjuvant, this adjuvant had strong antibody production-augmenting activity when compared to the control group and this could be realized with a very small amount.

It should be noted that when interferon β is administered as an antiviral drug or anticancer drug, proteinuria is one adverse effect that develops at high frequency when compared to administration of interferon α (non-patent reference 8).

Thus, there was so far no knowledge whatsoever during studies of cytokines as mucosal adjuvants that the family of interferon α, a cytokine for which there is an abundance of actual results in terms of use in humans, have mucosal adjuvant activity and can be used as a mucosal adjuvant; the adjuvant activity thereof is excellent; and when an interferon α is used as a mucosal adjuvant, both vaccine antigen-specific antibody in blood and vaccine antigen-specific antibody secreted from mucous membranes can be induced.

[Patent reference 1] Japanese Kokoku Patent No. H8-32633

[Patent reference 2] W099/44635 International Publication Pamphlet

[Patent reference 3] Japanese Kokokai Patent No. 2000-154148

[Non-patent reference 1] M. Takahashi et al., Drug Delivery System, Vol. 14

[Non-patent reference 2] R. K. Gupta et al., Vaccine, Vol. 13

[Non-patent reference 3] H. P. A. Hughes, Veterinary Immunology and Immunopathology, Vol. 63

[Non-patent reference 4] Victor, C. H. et al., International Immunopharmacology, 3(2003), 801-809

[Non-patent reference 5] Prosper, N. B., et al., Advanced Drug Delivery Reviews, 51(2001), 71-79

[Non-patent reference 6] Mariarosaria, M. et al., The Journal of Immunology, 162(1999), 114-121

[Non-patent reference 7] Vicente, C. et al., Journal of Hepatology, 32(2000), 317-324

[Non-patent reference 8] Kuramoto, I. et al., Acta Hepatologica Japonica, 33(1992), 517-523

### DISCLOSURE OF THE INVENTION

The inventors performed intense studies of methods of effectively inducing both antibodies in blood specific to various vaccine antigens and antibodies secreted at the mucous membrane surface specific to various vaccine antigens and selection of a mucosal adjuvant that can be used at this time and discovered that a family of several interferons α, which are normally used as anti-viral agents, and the like, have mucosal adjuvant activity, this activity as an adjuvant is excellent, and when an interferon α is used as a mucosal adjuvant, both vaccine antigen-specific antibody in blood and vaccine antigen-specific antibody secreted at mucous membranes can be induced. They further discovered that when an interferon α is used as a mucosal adjuvant, both antibodies in blood specific to various vaccine antigens and antibodies secreted at the mucosal surface specific to various vaccine antigens can be effectively induced by administration of the mucosal adjuvant by the same administration route as the mucosal administration route for the vaccine antigen, and as a result they completed the present invention.

That is, the present invention relates to “a method of inducing both vaccine antigen-specific antibody in blood and vaccine antigen-specific antibody secreted at the mucosal surface using vaccine antigen and adjuvant of this vaccine antigen, comprising

(1) mucosal administration of vaccine antigen,

(2) the use of an interferon α as the active ingredient of the adjuvant,

(3) administration of this adjuvant at the same time as or at a different time than this vaccine antigen, and

(4) mucosal administration of this adjuvant by the same administration route as this vaccine antigen.”

Moreover, the present invention relates to “a vaccine composition, comprising vaccine antigen and an interferon α as a mucosal adjuvant and which induces both vaccine antigen-specific antibody in blood and vaccine antigen-specific antibody secreted at the mucosal surface using vaccine antigen and adjuvant of this vaccine antigen by mucosal administration of this vaccine antigen and mucosal adjuvant at the same time or at different times and by the same route.”

The present invention further relates to “a mucosal adjuvant for inducing both vaccine antigen-specific antibody in blood and vaccine antigen-specific antibody secreted at the mucosal surface, comprising an interferon α as the active ingredient of this mucosal adjuvant and with which mucosal administration of this mucosal adjuvant is performed at the same time as or at a different time than this vaccine antigen and by the same route as the administration route for this vaccine antigen.”

The present invention yet further relates to “a combined product of a vaccine antigen and mucosal adjuvant for inducing both vaccine antigen-specific antibody in blood and vaccine antigen-specific antibody secreted at the mucosal surface, with which this mucosal adjuvant comprises an interferon α as the active ingredient and mucosal administration of this mucosal adjuvant is performed at the same time as or at a different time than this vaccine antigen and by the same route as the administration route for this vaccine antigen.

The present invention also independently relates to “a mucosal adjuvant for inducing both vaccine antigen-specific antibody in blood and vaccine antigen-specific antibody secreted at the mucosal surface, comprising an interferon α as the active ingredient,” “the use of interferon α for producing a mucosal adjuvant for inducing both vaccine antigen-specific antibody in blood and vaccine antigen-specific antibody secreted at the mucosal surface,” and “a mucosal immune response activation method, comprising administration of mucosal adjuvant containing interferon α as the active ingredient at the same time as or at a different time than the vaccine antigen and by the same administration route as the vaccine antigen to subjects in whom it is necessary to augment immunity by inducing both vaccine antigen-specific antibody in blood and vaccine antigen-specific antibody secreted at the mucosal surface.”

Various types of natural interferons α produced by macrophages also referred to as leukocyte interferons, a recombinant family of several interferons α, which are obtained by producing the gene-modified form of interferon from Escherichia coli, yeast, insect cells, animal-derived cells and the like in which these interferon α genes have been recombined and purifying this recombined form, consensus interferons α having the consensus sequence of various types of interferons α, for instance, interferon alfacon-1, and the like, can be used as the “family of several interferons α” cited in the present invention. Although there are no special restrictions, taking into consideration safety in humans, of these, commercial interferons α that are guaranteed to be safe are preferred.

The “vaccine antigen” cited in the present invention means mainly a protein or peptide antigen, and a vaccine antigen comprising a protein or peptide that has been produced from a protective antigen against infectious microorganisms, for instance influenza hemagglutinin A, can be given as an example. That is, there are no particular restrictions [to the vaccine antigen] as long as it is a protein or peptide component derived from an infectious microorganism or virus that can be the target of the vaccine. [Vaccine antigens] in which the toxin protein produced by an infectious microorganism has been inactivated, for instance, tetanus toxoid, pertussis vaccine, and inactivated vaccine in which live vaccine and the like have been inactivated, specifically polio, rubella, measles, rabies, influenza, HIV, hepatitis A vaccine, and the like, are included among these [vaccine antigens]. Vaccines that have been produced by technology such as recombination of the target antigen, and the like, for instance, lime disease vaccine and hepatitis B vaccine, can further be cited. In addition, it is also possible to include live vaccine antigen. For instance, Polio vaccine, rotavirus vaccine, cholera vaccine, tetanus vaccine, diphtheria vaccine, typhoid vaccine, E. coli vaccine, varicella vaccine, influenza vaccine, H. pylori vaccine, and the like can be cited as examples. One or a combination of two or more of these vaccine antigens can be used as needed.

The “vaccine composition” in the present invention means a composition of the various excipients listed below to the vaccine antigen that is prepared by various preparation methods. Moreover, the “mucosal adjuvant” in the present invention similarly can include compositions of various excipients added to an interferon α as the active ingredient prepared by various preparation methods.

In addition, “the combined product of vaccine antigen and mucosal adjuvant” is not necessarily in the form of a composition, although it can include compositions that comprise both components. It can also include preparations wherein the vaccine antigen and mucosal adjuvant are separate, such as a kit preparation that is prepared at the time of use.

There are no special restrictions to the amount per dose of the interferon α that is used as the adjuvant in the present invention as long as it is no more than the minimum dose generally used as an injection, but it is preferred that it is within a range of 0.5 to 5,000,000 IU. Within a range of 0.5 to 500,000 IU is further preferred and within a range of 0.5 to 10,000 IU is particularly preferred.

There are no special restrictions to the administration method for inducing a mucosal immune response of the present invention as long as it is a method of administration via the mucous membranes. Mucosal administration, such as nasal administration, oral administration, pulmonary administration, and vaginal administration is cited, the purpose being absorption and action in mucous membranes or lymphoid tissue present in mucous membranes. “Nasal administration” means administration through the nostrils, and nose drops and nasal spray can be given as examples of the pharmaceutical preparation. “Oral administration” means administration through the mouth in general, the buccal cavity, or the pharynx, and powders, fine particles, granules, tablets, capsules, pills, elixirs, syrup, troches, sublingual tablets, buccal tablets, and tablets that are quick-disintegrating in the buccal cavity can be cited as examples of the pharmaceutical preparation. “Pulmonary administration” means administration through the respiratory tract, and inhalations and sprays can be cited as examples of the pharmaceutical preparation. “Vaginal administration” means administration through the vagina, and vaginal suppositories, vaginal tablets, and sprays can be cited as examples of pharmaceutical preparations.

Moreover, during administration, it is necessary to administer the vaccine antigen and the interferon α to the same mucous membrane. However, as long as [the vaccine antigen and interferon α are administered to] the same mucous membrane, pre-administration, simultaneous administration, or post-administration is possible. “Pre-administration” means that the vaccine antigen is administered once the interferon α has been administered. “Simultaneous administration” means administration of the vaccine antigen and the interferon α to the above-mentioned mucous membranes at the same time, and a composition containing at least vaccine antigen and interferon α can be administered, or separate compositions can be simultaneously administered. “Post-administration” means that the interferon α is administered once the vaccine antigen has been administered. The “at a different time than” during administration indicates either the above-mentioned “pre-administration” or “post-administration.” The difference in this administration time is from one minute to twelve hours, preferably five minutes to six hours, ideally five minutes to four hours.

Appropriate excipients that are pharmaceutically acceptable can also be added to the interferon α or vaccine antigen to make a separate composition of each. The “combination” cited in the present invention means a combination of a composition containing the above-mentioned interferon α and a vaccine antigen or a composition containing a vaccine antigen and does not mean making the two into one composition.

In addition, it is also possible to add appropriate excipients that are pharmaceutically acceptable and make a composition containing at least both the interferon α and vaccine antigen.

The mixture ratio of vaccine antigen and interferon α when they are made into a composition containing both is difficult to consistently specify and should not be defined because it depends on the activity of the vaccine antigen that is used, and the like. However, the ratio of vaccine antigen is 0.01 to 55% w/w, preferably 0.05 to 50% w/w, particularly 0.1 to 45% w/w, of the entire composition, and the ratio of interferon α is 0.01 to 5% w/w, preferably 0.05 to 4% w/w, particularly 0.1 to 2.5% w/w, of the entire composition.

The “antigen-specific antibody in blood” cited in the present invention means immunoglobulin produced only to a specific antigen and induced in blood. It is known that in general, there are five classes of immunoglobulin based on different amino acid sequences of their H chain (IgM, IgG, IgA, IgD, and IgE). Of these, IgG antibody bears the main burden for acquired immunity and is the immunoglobulin that is the most abundant in blood. Moreover the “antigen-specific antibody secreted at the mucosal surface” means immunoglobulin produced only to a specific antigen and secreted at the mucosal surface. In general, mainly secreted IgA antibody is known. IgA antibody usually forms a dimer and is secreted on the mucosal surface and widely distributed over the mucosal surface via receptors that are present in mucosal epithelial cells in the trachea, intestines, salivary glands, and the like. Consequently, with respect to the “antigen-specific antibody in blood” and “antigen-specific antibody secreted at the mucosal surface,” of the present invention, the IgG present in blood and IgA present in mucous membranes (for instance, the IgA in feces) are determined, respectively.

Moreover, salts, surfactants, saccharides, amino acids, organic acids, and other water-soluble substance are cited as examples of excipients that are pharmaceutically acceptable, and one or two or more of these can be added. Potassium L-glutamate, sodium L-glutamate, sodium edetate, sodium caprylate, carbazochrome sodium sulfonate, carboxymethylcellulose sodium, sodium citrate, calcium gluconate, sodium gluconate, magnesium gluconate, sodium metasulfobenzoate, sodium monohydrogen phosphate, sodium dihydrogen phosphate, dipotassium phosphate, potassium dihydrogen phosphate, aluminum chloride, potassium chloride, calcium chloride, sodium chloride, sodium acetate, sodium carbonate, sodium bicarbonate, and the like are cited as specific salts. D-sorbitol, D-mannitol, inositol, xylitol, dextran, glucose, maltose, lactose, sucrose, and the like are cited as saccharides. Methionine, aspartic acid, alanine, arginine, glycine, cystein, taurine, histidine, phenylalanine, glutamic acid, lysine, and the like are cited as amino acids. Ascorbic acid, human serum albumin, chondroitin sodium sulfate, gelatin, gelatin hydrolysate, heparin sodium, and the like are cited as other water-soluble substances. Moreover, surfactants, organic acids, and the like can be added. Sorbitan sesquioleate, sorbitan fatty acid ester, polyoxyethylene (160) polyoxypropylene (30) glycol, polyoxyethylene sorbitan monolaurate, polyoxyethylene castor oil, polyoxyethylene hydrogenated castor oil 50, polyoxyethylene hydrogenated castor oil 60, polysorbate 20, polysorbate 80, Macrogol 400, Macrogol 4000, Macrogol 600, and the like are cited as surfactants, and oleic acid, thioglycolic acid, lactic acid, and the like are cited as organic acids. Moreover, pH-regulating agents, such as hydrochloric acid and sodium hydroxide, and osmotic pressure-regulating agents, such as sodium chloride, can be added as needed.

It is also possible to add excipients other than the above-mentioned that are pharmaceutically acceptable. One or two or more are selected in appropriate amounts from the fillers of potato starch, wheat starch, rice starch, corn starch, crystalline cellulose, and the like, the binders of hydroxypropylmethyl cellulose, hydroxypropyl cellulose, methyl cellulose, gum arabic, and the like, the swelling agents of carboxymethyl cellulose, carboxymethylcellulose calcium, croscarmellose sodium, and the like, the lubricants of stearic acid, calcium stearate, magnesium stearate, talc, magnesium metasilicate, magnesium aluminate [Tr's note: listed as a single chemical in Japanese original], calcium hydrogen phosphate, anhydrous calcium hydrogen phosphate, and the like, the fluidizing agents of hydrous silicon dioxide, light silicic anhydride, dry aluminum hydride gel, and the like, the coloring agents of yellow ferric oxide, red ferric oxide, and the like, the coating agents of zein, hydroxypropylmethyl cellulose, hydroxypropyl cellulose, and the like, the fragrances of 1-menthol, mint oil, fennel oil, and the like, the preservatives of sodium sorbate, potassium sorbate, methyl parabenzoate, ethyl parabenzoate, and the like, the buffers of citric acid, succinic acid, fumaric acid, tartaric acid, magnesium oxide, zinc oxide, magnesium hydroxide, phosphoric acid, boric acid, their salts, and the like.

The above-mentioned excipients are not limited to those given as examples, and there are no special restrictions to the amount added as long as it is normally used pharmaceutically by persons in the field and it is within a range that will not compromise the results of the invention.

Other adjuvants, such as aluminum phosphate, aluminum hydroxide, and calcium phosphate can be included as long as they are within a range that will not compromise the results of the present invention.

A composition can be easily prepared as an aqueous solution by adding and mixing an interferon α with a vaccine that has been used in the past. Moreover, it is also possible to add excipients, such as filler or stabilizer, as needed to vaccine antigen solution and aqueous interferon α solution and then made this into a powder by a method such as spray drying or freeze drying. The composition comprising vaccine antigen and interferon α obtained in this way can be used for mucosal administration in this form.

Moreover, the vaccine composition of the present invention can also be used after being encapsulated as needed in a drug delivery system (DDS), such as a liposome, nanosphere, or microsphere, or a carrier, such as a biodegradable carrier or mucoadhesive carrier. Specific examples are compositions of vaccine antigen and interferon α encapsulated in a microsphere comprising a biodegradable polymer, such as PLGA and a composition of vaccine antigen and interferon α encapsulated in mucoadhesive microspheres comprising hyaluronic acid, chitin, and the like. Moreover, efficient and effective technology for administration to the mucosal lymphoid tissue can be used, for instance, technology for targeting M cells or Peyer's patches that exist in the gastrointestinal mucosal lymphoid tissue, specifically technology whereby vaccine antigen and interferon α are encapsulated in microspheres having M cell-specific lectin and antibody molecules on the surface, and the like as the carrier. In addition, it is also possible to use recently reported technology that employs detoxified bacteria or virus itself, such as inactivated Salmonella, as the carrier.

Moreover, the present invention is useful as a method of inducing mucosal immune response in not only humans, but also animals. When used in animals, it is preferred that interferon α of the animal species in question be used, but there are no special restrictions. Human interferon α can also be used in animals that show human interferon α cross ability, that is, reactivity to human interferon α.

### Preferred Embodiments

The present invention will now be described further while citing examples, but the present invention is not limited to these examples.

### EXAMPLE 1

Mouse nasal administration was performed in accordance with the method of Yamamoto et al (S. Yamamoto et al., Proc. Natl. Acad. Sci. USA, Vol. 94, 1997) using ovalbumin (OVA hereafter), which is widely used in literature as a model antigen. Four or five C57BL mice (male, 8 weeks old) per group were used. The interferon α was mouse interferon α, and it was administered at the same time as the antigen (100 μg/mouse OVA) at 1.5 μg (6,500 U)/mouse per dose. Administration was always nasal administration. After administration a total of three times, the initial administration and one week and two weeks after [the initial administration], blood was sampled 3 weeks, 4 weeks, and 6 weeks from the day of the initial administration. The blood was centrifuged for 15 minutes at 3,000 rpm and the supernatant was recovered. The OVA-specific antibody titer in these serum samples (blood IgG) was assayed by the ELISA method (Table 1).

### COMPARATIVE EXAMPLE 1

OVA was administered at 100 μg/mouse per dose to five C57BL mice (male, 8 weeks old) per group. Administration was always nasal administration. After administration a total of three times, the initial administration and one week and two weeks after [the initial administration], blood was sampled 3 weeks, 4 weeks, and 6 weeks from the day of the initial administration. The blood was centrifuged for 15 minutes at 3,000 rpm and the supernatant was recovered. The OVA-specific antibody titer in these serum samples (blood IgG) was assayed by the ELISA method (Table 1).

TABLE 1
OVA-specific blood IgG titer in Example
1 and Comparative Example 1

OVA-specific blood IgG titer (OD 490 nm)

| | Comparative Example 1 | Example 1 |
|---|---|---|
| 3 w | 0.13 | 0.41 |
| | 0.19 | 0.85 |
| | 0.31 | 0.87 |
| | 0.51 | 1.46 |
| | 0.73 | |
| 4 w | 0.16 | 0.69 |
| | 0.23 | 0.95 |
| | 0.32 | 1.01 |
| | 0.33 | 1.08 |
| | 0.61 | |
| 6 w | 0.11 | 0.49 |
| | 0.12 | 0.83 |
| | 0.28 | 0.89 |
| | 0.44 | 1.01 |
| | 0.45 | 1.10 |

Discussion

As is clear from Table 1, the interferon α concomitant use group showed a significantly high OVA-specific blood IgG titer when compared to Comparative Example 1 during all weeks. These results indicate that a systemic immune response can be effectively induced by nasal administration of vaccine concomitant with interferon α.

### EXAMPLE 2

As in example 1, OVA was administered at 100 μg/mouse per dose to four or five C57BL mice (males, 8 weeks old) per group. Interferon α was administered at the same time as the antigen at 1.5 μg/mouse. Administration was always nasal administration. After administration a total of three times, the initial administration and one week and two weeks after [the initial administration], an approximately one-day sample of feces was collected beginning the day before the third week, fourth week, and sixth week from the day of the initial administration. Exactly 250 mg of this feces sample were weighed out, 1 ml of Tris hydrochloride buffer (pH of 7.4) was added and stirred, and then this was centrifuged for 15 minutes at 3,000 rpm and the supernatant was recovered. The OVA-specific antibody titer in the supernatant (fecal IgG) was assayed by the ELISA method (Table 2).

### COMPARATIVE EXAMPLE 2

OVA was administered at 100 μg/mouse per dose to five C57BL mice (males, 8 weeks old) per group. Administration was always nasal administration. After administration a total of three times, the initial administration and one week and two weeks after [the initial administration], an approximately one-day sample of feces was collected beginning the day before the third week and fourth week from the day of the initial administration. Exactly 250 mg of this feces sample were weighed out, 1 ml of Tris hydrochloride buffer (pH of 7.4) was added and stirred, and then this was centrifuged for 15 minutes at 3,000 rpm and the supernatant was recovered. The OVA-specific antibody titer in the supernatant (fecal IgG) was assayed by the ELISA method (Table 2).

TABLE 2
OVA-specific fecal IgA titer in Example
2 and Comparative Example 2

OVA-specific fecal IgA titer (OD 490 nm)

| | Comparative Example 2 | Example 2 |
|---|---|---|
| 3 w | 0.16 | 0.74 |
| | 0.51 | 0.85 |
| | 0.55 | 0.86 |
| | 0.61 | 0.87 |
| | 0.76 | |
| 4 w | 0.46 | 0.62 |
| | 0.57 | 1.09 |
| | 0.71 | 1.12 |
| | 0.76 | 1.27 |
| | 2.34 | |

Discussion

As shown in Table 2, Example 2 showed a significantly high OVA-specific fecal IgA titer when compared to Comparative Example 3, particularly during the third and fourth weeks, and therefore, an immune response was induced on the gastrointestinal mucous membranes by concomitant use of interferon α by nasal administration. These results indicate that a systemic immune response as well as a mucosal immune response can be induced by concomitant use of interferon α by nasal administration.

### INDUSTRIAL APPLICABILITY

The present invention makes it possible to induce both vaccine antigen-specific antibody in blood and vaccine antigen-specific antibody secreted on the mucous membrane using a vaccine antigen and adjuvant of this vaccine antigen, produce a mucosal vaccine that provides better results than in the past, and provide a technology that is effectively prevents infection.

1. A method of inducing both vaccine antigen-specific antibody in blood and vaccine antigen-specific antibody secreted at the mucosal surface using vaccine antigen and adjuvant of this vaccine antigen, comprising
(1) mucosal administration of vaccine antigen,
(2) the use of an interferon α as the active ingredient of the adjuvant,
(3) administration of said adjuvant at the same time as or at a different time than said vaccine antigen, and
(4) mucosal administration of said adjuvant by the same administration route as said vaccine antigen.

2. The method according to claim 1, wherein the interferon α is selected from natural interferons a and recombinant interferons α.

3. The method according to claim 2, wherein the amount of interferon α used is 0.5 to 5,000,000 IU.

4. The method according to claim 2, wherein the vaccine antigen is protein or peptide antigen.

5. The method according to claim 2, wherein mucosal administration is performed at the same time.

6. The method according to claim 5, wherein administration is via the nasal mucous membrane.
